(19) United States
(12) Patent Application Publication
(10) Pub. No.: US 2022/0110647 A1
(43) Pub. Date: Apr. 14, 2022

(54) Thrombus Aspiration Systems and Related Methods

(71) Applicant: ASAHI INTECC CO., LTD., Seto-shi (JP)

(72) Inventors: Bruce Wilson, Temecula, CA (US); Michael Luk, Orange, CA (US)

(73) Assignee: ASAHI INTECC CO., LTD., Seto-Shi (JP)

(21) Appl. No.: 17/498,517

(22) Filed: Oct. 11, 2021

Related U.S. Application Data
(60) Provisional application No. 63/091,257, filed on Oct. 13, 2020.

(51) Int. Cl. A61B 17/221

(52) U.S. Cl. CPC A61B 17/221; A61B 2017/22038

(57) ABSTRACT

Systems for use in blood clot removal can comprise a receiver-supporting element, a self- expanding receiver that includes a frame and a polymer, a first tube connected to the receiver, and a second tube positioned around at least a portion of the receiver. The receiver-supporting element can have a distal end, a proximal end, and a lumen extending proximally from the distal end. At least a portion of the self-expanding receiver can be positioned around at least a portion of a first segment of the receiver-supporting element and the first tube can be positioned around at least a portion of a second segment of the receiver-supporting element. The second tube can include separable portions.

### CROSS-REFERENCE TO RELATED APPLICATIONS

This application claims priority to U.S. Provisional Application No. 63/091,257, entitled “THROMBUS ASPIRATION SYSTEMS AND RELATED METHODS,” filed Oct. 13, 2020, the content of which is incorporated by reference in its entirety.

### FIELD OF INVENTION

The present invention relates generally to systems for thrombus removal and, more specifically, to systems suitable for removing thrombi via aspiration.

### BACKGROUND

A thrombus (also referred to as a blood clot) can block the flow of blood through a vessel, thereby depriving tissues of blood and oxygen and causing damage thereto. Thrombi are the predominant cause of strokes, which require prompt treatment to mitigate the risk of long-term disability and death.

A thrombectomy is a common procedure for treating strokes. In a thrombectomy, a guide catheter is inserted into a patient's vasculature at the groin and advanced therethrough toward the thrombus. A stent retriever can then be passed through the guide catheter and engage the thrombus to capture it; once the thrombus is captured, the stent retriever and catheter can be removed to restore blood flow to the brain. Alternatively, a small-bore aspiration catheter can be passed through the guide catheter and, when its distal end is at the thrombus, a vacuum can be applied at the catheter's proximal end to draw the thrombus against the aspiration catheter's mouth for removal. Over the past decade, thrombectomies have improved the stroke treatment success rate, with about 85% of the procedures achieving recanalization.

However, the inventor has recognized a number of challenges that have prevented successful recanalization in all thrombectomies. For example, blood flow against a thrombus acts to impede removal. While some have attempted to address this with a balloon guide catheter at the internal carotid artery (ICA) that blocks ICA blood flow to the thrombus, other vessels can continue to supply blood to the neurovasculature and thereby continue to impede thrombus removal.

Additionally, thrombi include (1) white thrombi that predominantly comprise platelets and (2) red thrombi that predominantly comprise red blood cells. These different thrombi compositions yield different mechanical properties, with white thrombi tending to have a higher Young's modulus and tensile strength and red thrombi tending to have a lower Young's modulus and tensile strength. Accordingly, stent retrievers can readily achieve mechanical engagement with red thrombi for removal, but may not be able to capture white thrombi, which are harder than red thrombi.

Aspiration catheters can maintain a hold of thrombi at the mouth thereof when the vacuum is applied, even if a stent retriever would not be able to mechanically engage such thrombi. But aspiration catheters face challenges as well. Because aspiration catheters must be able to access the vasculature in which a thrombus is located—commonly the ICA or the middle cerebral artery (MCA) (e.g., the M1 segment thereof), for strokes—they usually are relatively narrow, having a diameter that is less than 50% of the diameter of the blood vessel. Such narrow aspiration catheters may not be able to ingest a stroke-inducing thrombus that spans across the blood vessel. As a result, thrombus removal is often achieved by retracting the aspiration catheter with most of the thrombus disposed outside of its lumen, rather than by allowing the vacuum source to draw the thrombus through the lumen. The exposed thrombus is at risk of detachment during catheter withdrawal, which can result in failed recanalization.

Some have improved aspiration success rates with larger-diameter aspiration catheters whose design allows them to reach the target vasculature despite their size. For example, while aspiration catheters commonly had an internal diameter of 0.066″, MicroVention, Inc. developed the SOFIA™ Plus catheter that has a 0.070″ internal diameter but can reach the MCA, and Perfuze Ltd. is developing the Millipede CIS catheter that has an internal diameter of 0.088″. While these aspiration catheters have larger lumen cross-sectional areas and thus can yield larger suction forces during aspiration, they still may not be able to ingest thrombi, which often have a diameters that are around twice as large (e.g., around 0.157″).

Another approach to aspiration includes advancing a self-expanding stent disposed within a small-diameter sheath to a thrombus and deploying the stent distally out of the sheath such that a distal portion of the stent expands radially to the artery wall. In this manner, the stent can ingest the thrombus through its expanded mouth. One example of such a device is the Anaconda Biomed S.L. ANCD Advanced Thrombectomy System. While such devices can have an expanded mouth through which a thrombus may readily pass, their stent narrows at a proximal portion thereof to a diameter than is smaller than that of the sheath to which the stent is attached. Because these devices typically use multiple telescopic catheters to reach the ICA or MCA, with larger-diameter catheters positioned proximally for rigidity and smaller-diameter catheters extending from the larger-diameter catheters for distal flexibility to allow the device to navigate the vasculature, the constriction can be relatively narrow. For example, the stent of the ANCD Advanced Thrombectomy System narrows down to a 0.043″ internal diameter. This constriction can impede ingestion of the thrombus through the sheath during aspiration.

Accordingly, there is a need in the art for thrombectomy systems that can better ingest thrombi to increase the likelihood of successful recanalization.

### SUMMARY

The present systems address this need in the art with a self-expanding receiver, a first tube connected to the receiver, a receiver-supporting element, and a second tube positioned around at least a portion of the receiver such that the receiver is compressed from its expanded state. The receiver-supporting element can be used to advance the first tube and receiver within a guide catheter and toward a thrombus by engaging the first tube when a distally-urging force is applied to the receiver-supporting element. Additionally, the receiver-supporting element can have first and second segments, where at least a portion of the receiver is positioned around the first segment and at least a portion of the first tube is positioned around the second segment. With the first and second segments position within the receiver and first tube, the first tube and compressed receiver can have relatively large cross-sectional dimensions and the system can have enough flexibility in a distal portion thereof to reach a thrombus in the ICA or MCA. The receiver-supporting element can also have a third segment that is positioned proximal of and has a larger cross-sectional dimension than the first and second segments to define a shoulder portion configured to engage the first tube to advance the first tube and the receiver. The thicker third segment can promote the rigidity of a proximal portion of the system to facilitate delivery of the first tube and receiver to the thrombus.

The self-expanding receiver can be deployed such that a distal portion thereof radially expands to engage the vessel wall, thereby yielding a large mouth that can readily ingest the thrombus during aspiration. Because the receiver-supporting element permits the first tube—and thus the receiver's throat—to have a relatively large inner cross-sectional dimension (e.g., at least 0.075″), the thrombus can more readily be ingested into the first tube during aspiration, thereby promoting higher successful recanalization rates.

Some of the present systems for blood clot removal include a receiver-supporting element, a self-expanding receiver, a first tube connected to the receiver, and a second tube positioned around at least a portion of the receiver. Some of the present methods comprise advancing a guidewire through vasculature of the patient, advancing a catheter over the guidewire, and advancing a system over the guidewire and into the catheter. In some of such methods, the system comprises a receiver-supporting element, a self-expanding receiver, a first tube connected to the receiver, and a second tube positioned around at least a portion of the receiver.

In some embodiments, the receiver-supporting embodiment has a first segment that includes a first segment cross-sectional outer dimension and a second segment that includes a second segment cross-sectional outer dimension. The first segment cross-sectional outer dimension and the second segment cross-sectional outer dimension, in some embodiments, are the same. In other embodiments, the second segment cross-sectional outer dimension is greater than the first segment cross-sectional outer dimension. At least a portion of the receiver, in some embodiments, is positioned around at least a portion of the first segment or around a first portion of the receiver-supporting element. At least a portion of the first tube, in some embodiments, is positioned around at least a portion of the second segment or around a second portion of the receiver-supporting element.

In some embodiments, the receiver-supporting element has a distal end, a proximal end, and a lumen extending proximally from the distal end and through at least the first and second segments of the receiver-supporting element. The receiver-supporting element, in some embodiments, has an atraumatic distal tip having a cross-sectional outer dimension larger than the first segment cross-sectional outer dimension. In some of such embodiments, the atraumatic distal tip has a region in which a distal end of the receiver is positioned.

In some embodiments, the system comprises a positioning element connected to the first tube. The positioning element, in some embodiments, comprises a push rod that comprises metal. In some embodiments, the positioning element comprises non-metal and is non-rigid. The receiver-supporting element, in some embodiments, has a channel, wherein optionally a portion of the positioning element is positioned in at least a portion of the channel. In some embodiments, the receiver-supporting element has a third segment or a third portion positioned proximal of the first and second segments, the third segment or third portion optionally including the channel. The third segment, in some embodiments, has a cross-sectional outer dimension larger than the second segment cross-sectional outer dimension. The third segment, in some embodiments, has shoulder portion no more than five centimeters away from a proximal end of the first tube. In some embodiments, the system comprises a shoulder support connected to both the first tube and the positioning element. In some embodiments, a proximal portion of the receiver-supporting element has a durometer greater than a durometer of a distal portion of the receiver-supporting element.

The receiver-supporting element, in some embodiments, also has a hub. In some embodiments, a proximal end of the second tube is distal of the proximal end of the receiver-supporting element. The receiver-supporting element, in some embodiments, also has a connector proximal of the hub.

In some embodiments, the self-expanding receiver includes a frame and a polymer. At least a portion of the frame, in some embodiments, is surrounded by at least a portion of the polymer. The receiver, in some embodiments, has an outer hydrophilic coating. The frame, in some embodiments, comprises a braid. In some embodiments, the receiver also has a radiopaque marker positioned no more than one centimeter from a distal end of the receiver.

The first tube, in some embodiments, includes a frame, a polymer, and an inner liner. In some embodiments, the frame of the first tube and the frame of the receiver are in direct contact with each other. In some embodiments, the positioning element is connected to the first tube through a ring, wherein optionally at least a portion of the ring is surrounded by at least a portion of the polymer of the first tube.

The second tube, in some embodiments, has separable portions. In some embodiments, the second tube is also positioned around at least a portion of the first tube. The second tube, in some embodiments, also has a valve seal comprising separable valve seal portions. In some embodiments, the second tube has a proximal end, a distal end, and a length from the proximal end to the distal end. The length, in some embodiments, is at least 110 cm. In other embodiments, the length is less than or equal to 20 cm.

In some embodiments, the receiver-supporting element, the self-expanding receiver, the first tube, the second tube, and/or the positioning element are positioned in a sealed container.

Some methods comprise advancing the receiver-supporting element, the self-expanding receiver, the first tube, and the positioning element within the catheter while the second tube is not advanced over the guidewire. Other methods comprise advancing the receiver-supporting element, the self-expanding receiver, the first tube, the positioning element, and the second tube through at least a portion of the catheter until a distal end of the second tube is distal of a distal end of the catheter.

Some methods further comprise retracting the second tube out of the catheter and/or separating the separable portions of the second tube and removing the second tube from being positioned around the guidewire. In some of such methods, the method further comprises after the separating, applying force to the receiver-supporting element and/or the positioning element so as to advance the receiver-supporting element, the self-expanding receiver, the first tube, and the positioning element within the catheter. Some methods comprise, after the separating, applying force to the receiver-supporting element so as to advance the receiver-supporting element, the self-expanding receiver, the first tube, and the positioning element within the catheter.

Some methods comprise deploying the self-expanding receiver such that a distal portion of the self-expanding receiver expands and contacts a vessel of the patient. In some of such methods, the deploying comprises retracting the second tube while applying force to at least one of the positioning element and the receiver-supporting element. In some methods, the positioning element is rigid and the deploying comprises retracting the second tube while applying force to the positioning element and the receiver-supporting element. In other methods, the positioning element is non-rigid and the deploying comprises retracting the second tube while applying force to the receiver-supporting element.

Some methods comprise withdrawing the receiver-supporting element such that a distal end thereof is proximal of a proximal end of the first tube. Some methods comprise applying a vacuum to the catheter to help draw a blood clot into the distal portion of the self-expanding receiver. Some methods comprise pulling on the positioning element so that the first tube and the self-expanding receiver move proximally relative to the catheter.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed embodiment, the terms “substantially” and “approximately” may be substituted with “within [a percentage] of” what is specified, where the percentage includes 0.1, 1, 5, and 10 percent.

The terms “comprise” and any form thereof such as “comprises” and “comprising,” “have” and any form thereof such as “has” and “having,” and “include” and any form thereof such as “includes” and “including” are open-ended linking verbs. As a result, a product or system that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Each dimension herein provided in an English unit may be translated to the corresponding metric unit by rounding to the nearest millimeter.

Any embodiment of any of the products, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a side view of one of the present systems for removing a thrombus that includes a receiver, a first tube connected to the receiver, a positioning element connected to the first tube, a receiver-supporting element, and a second tube.

FIG. 1B depicts the system of FIG. 1A in an insertion configuration in which at least a portion of each of the receiver-supporting element, the first tube, and the self-expanding receiver are positioned within the second tube, with the receiver being compressed by the second tube.

FIG. 1C is a cross-sectional view of the system of FIG. 1A taken along line 1C-1C of FIG. 1B.

FIG. 1D is an enlarged, partial cross-sectional view of the system of FIG. 1A.

FIGS. 1E-1G are cross-sectional views of the system of FIG. 1A taken along lines 1E-1E, 1F-1F, and 1G-1G, respectively, of FIG. 1C.

FIG. 2A is a side view of the system of FIG. 1A extending through a guide catheter.

FIG. 2B is a cross-sectional view of the system of FIG. 1A extending through a guide catheter taken along line 2B-2B.

FIG. 3 is an enlarged, partial cross-sectional view of a second embodiment of the present systems in which a first segment of the receiver-supporting element is narrower than a second segment thereof and a tip of the receiver-supporting element includes a region configured to receive the distal end of the receiver.

FIG. 4A is a cross-sectional view of a third embodiment of the present systems in which the lumen of the receiver-supporting element extends from the distal end of the receiver-supporting element to a portion of the receiver-supporting element's third segment that is distal of the receiver-supporting element's proximal end, where the third segment includes a slit through which a guidewire extending through the lumen can exit the receiver-supporting element.

FIG. 4B is a cross-sectional view of a fourth embodiment of the present systems in which the lumen of the receiver-supporting element extends from the distal end and through the first, second, and third segments of the receiver-supporting element, where the receiver-supporting element includes a fourth segment that has a smaller outer cross-sectional dimension than the third segment such that a guidewire extending through the lumen can exit the receiver-supporting element without passing through the fourth segment.

FIG. 4C is a cross-sectional view of a fifth embodiment of the present systems in which a pusher extends proximally from a proximal end of the receiver-supporting element.

FIG. 5 is an enlarged, partial cross-sectional view of a sixth embodiment of the present systems that includes a shoulder support connected to the first tube and the positioning element and is configured to engage with a shoulder portion of the receiver-supporting element defined by a third segment thereof

FIG. 6 is a cross-sectional view of the system of FIG. 1A when the separable portions of the second tube are separated and the receiver is expanded.

FIG. 7 is a side view of a receiver and first tube that are suitable for use in some of the present systems, each including a polymer and a frame.

FIG. 8A is a side view of a multi-port adapter having a first port that is coupled to a proximal connector of a guide catheter, a second port through which the system of FIG. 1A is disposed and which forms a seal around the second tube, and a third port connected to a syringe for aspiration.

FIG. 8B is a side view of the multi-port adapter of FIG. 8A, with the system of FIG. 4C rather than the system of FIG. 1A disposed through the second port.

FIG. 8C is a side view of the multi-port adapter of FIG. 8A, with a vacuum pump rather than a syringe connected to the third port.

FIG. 9 is a side view of a kit in which the system of FIG. 1A is disposed within a sealed container.

FIG. 10A depicts vasculature of a patient with a thrombus positioned in the M1 segment of the middle cerebral artery, a guidewire extending to the thrombus, and a guide catheter extending to the bottom of the internal carotid artery.

FIG. 10B depicts the vasculature of FIG. 10A, where the second tube of the FIG. 1A system is disposed within and extends beyond the guide catheter and to the thrombus.

FIG. 11A is a partial cross-sectional view of the FIG. 1A system disposed in the FIG. 10A vasculature, where the system is in the insertion configuration such that the distal ends of the second tube and the receiver are positioned near the thrombus.

FIG. 11B is a partial cross-sectional view of the FIG. 1A system disposed in the FIG. 10A vasculature, where the receiver is deployed such that a distal portion thereof is expanded to the vessel wall.

FIG. 11C is a partial cross-sectional view of the FIG. 1A system disposed in the FIG. 10A vasculature, where the receiver-supporting element is withdrawn from the guide catheter and the receiver's distal end is in contact with the thrombus.

FIG. 12A is a partial cross-sectional view of the guidewire extending to the thrombus and the guide catheter positioned around a portion of the guidewire in the FIG. 10A vasculature.

FIG. 12B is a partial cross-sectional view of the FIG. 1A system positioned within a proximal portion of the guide catheter of FIG. 12A.

FIG. 12C is a partial cross-sectional view of the FIG. 1A system positioned within a proximal portion of the guide catheter of FIG. 12A with the second tube withdrawn from the guide catheter such that the receiver is expanded to the guide catheter's inner wall.

FIG. 12D is a partial cross-sectional view of the FIG. 1A system advanced to a distal portion of the guide catheter of FIG. 12A after the second tube is withdrawn.

FIG. 12E is a partial cross-sectional view of the FIG. 1A system with the receiver deployed from the guide catheter.

FIG. 13A is a partial cross-sectional view of the FIG. 1A system disposed in the FIG. 10A vasculature, where a vacuum is applied to the guide catheter while the receiver is deployed such that the thrombus moves within the receiver and to its transition section.

FIG. 13B is a partial cross-sectional view of the FIG. 1A system disposed in the FIG. 10A vasculature, where the thrombus is drawn into the first tube as the vacuum is applied to the guide catheter.

FIG. 13C is a partial cross-sectional view of the FIG. 1A system disposed in the FIG. 10A vasculature, where the thrombus is drawn into the guide catheter's lumen as the vacuum is applied to the guide catheter.

FIG. 13D is a partial cross-sectional view of the FIG. 1A system disposed in the FIG. 10A vasculature, where the first tube and receiver and withdrawn into the guide catheter.

### DETAILED DESCRIPTION

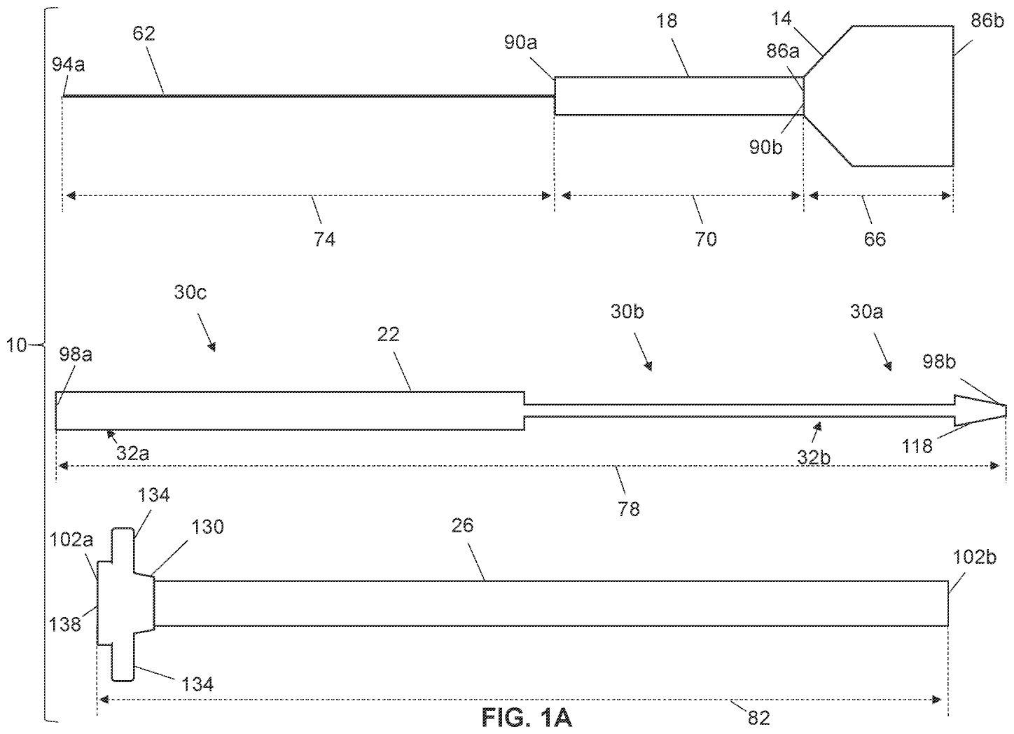

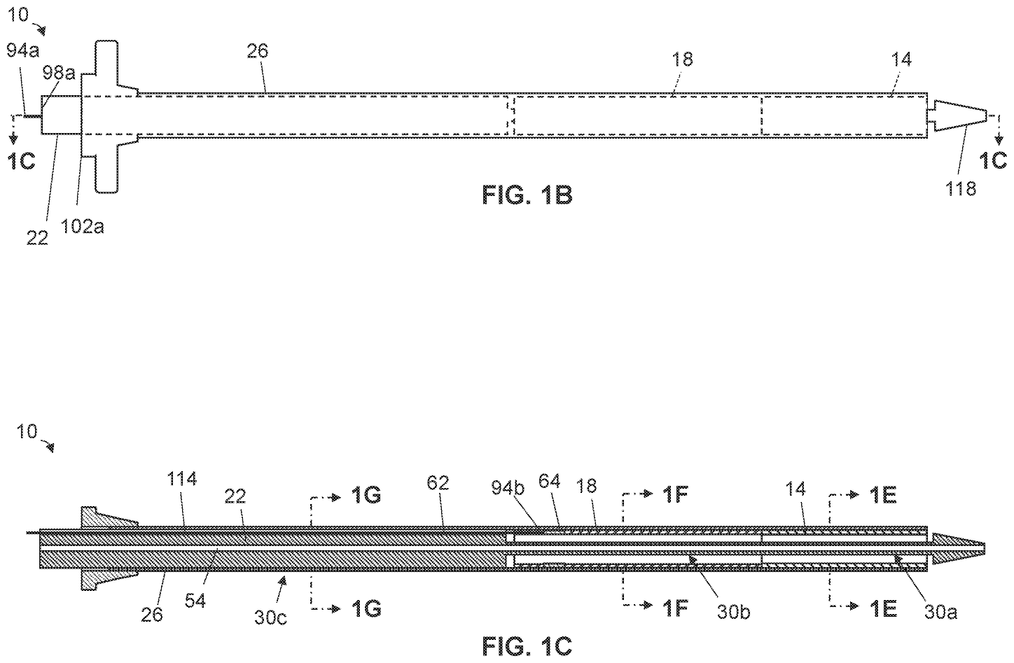

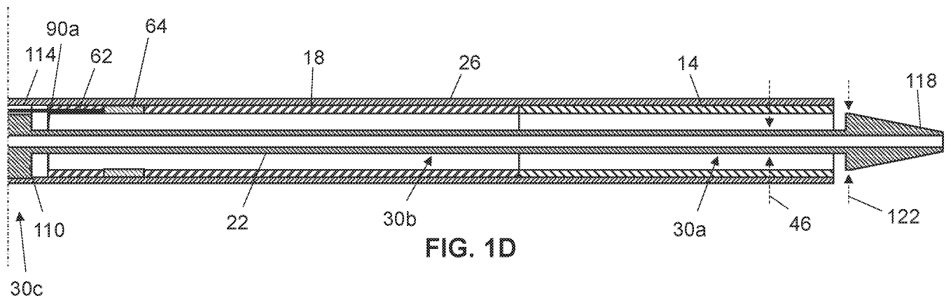

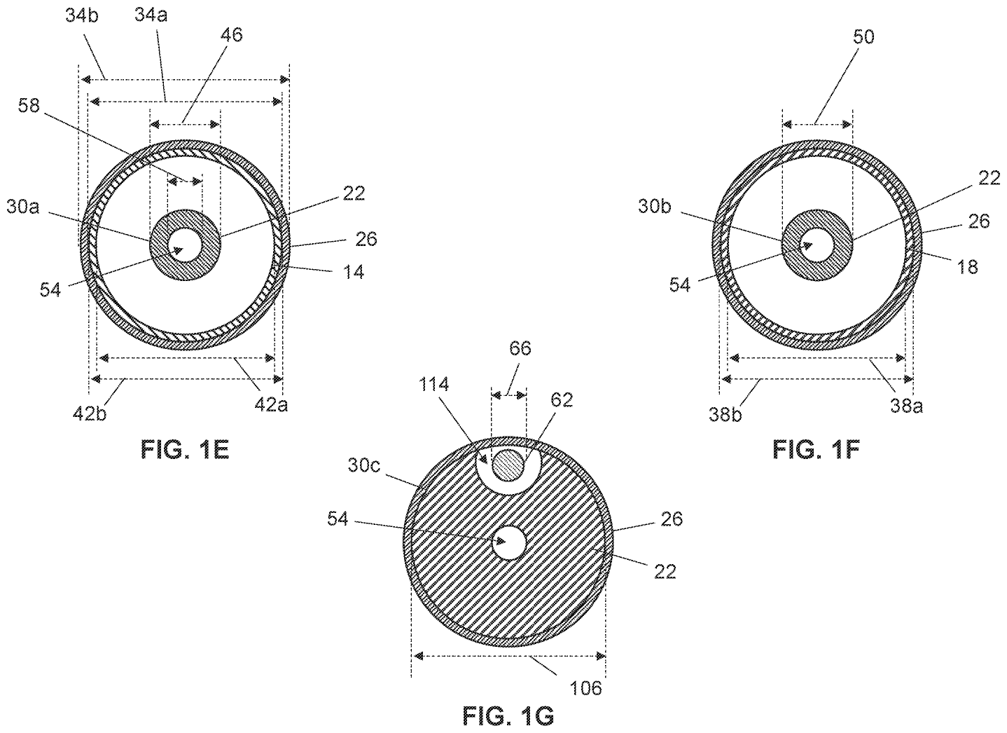

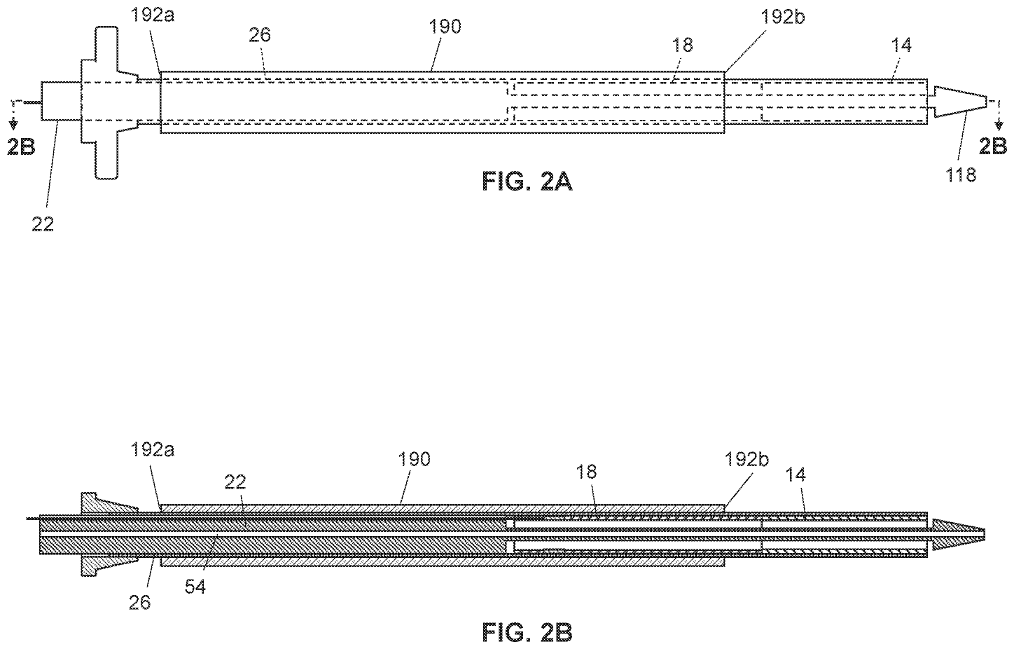

Referring to FIGS. 1A-1G, shown is a first embodiment 10 of the present systems for thrombus removal that includes a self-expanding receiver 14, a first tube 18 connected to the receiver, a receiver-supporting element 22, and a second tube 26. Second tube 26 can comprise a catheter or sheath defining a lumen that extends between its proximal and distal ends 102a and 102b such that, as shown in FIGS. 1B-1D, receiver 14, first tube 18, and receiver-supporting element 22 are each disposable at least partially within the second tube's lumen. With receiver 14, first tube 18, and receiver-supporting element 22 at least partially disposed in second tube 26, the receiver can be compressed from its expanded state (FIG. 1A) and, as illustrated in FIGS. 2A and 2B, system 10 can be inserted into a guide catheter 190 (e.g., an 8F catheter) that can be positioned within a patient's vasculature. In this manner, and as described in further detail below, receiver 14 and first tube 18 can be advanced through the vasculature along guide catheter 190 and to a thrombus that is positioned within, for example, the ICA or MCA (e.g., in the M1 segment thereof).

Receiver-supporting element 22 can facilitate advancement of receiver 14 and first tube 18 through the patient's vasculature, while allowing the receiver and first tube to have relatively large inner cross-sectional dimensions to facilitate thrombus aspiration therethrough. Referring particularly to FIGS. 1C-1G, receiver-supporting element 22 can include first, second, and/or third segments 30a, 30b, and 30c. At least a portion of receiver 14 and at least a portion of first tube 18 can be positioned around at least a portion of first and second segments 30a and 30b, respectively (FIGS. 1E and 1F). Third segment 30c can be positioned proximally of first and second segments 30a and 30b and can have an outer cross-sectional dimension (e.g., diameter) 106 that is larger than the first and second segments' outer cross-sectional dimensions (e.g., diameters) 46 and 50 (FIG. 1G). For example, third segment 30c's outer cross-sectional dimension 106 can be greater than or equal to any one of, or between any two of, 110%, 125%, 150%, 175%, 200%, or 300% of outer cross-sectional dimensions 46 and 50 of first and second segments 30a and 30b. In this manner, third segment 30c can include a shoulder portion 110 that is positioned within 5 cm, 4 cm, 3 cm, 2 cm, 1 cm, or 0.50 cm (e.g., is in contact with) the first tube's proximal end 90a and can engage the proximal end when a force urging the receiver-supporting element distally is applied thereto. Such engagement can cause receiver 14 and first tube 18 to advance through a patient's vasculature (e.g., within and beyond guide catheter 190).

Positioning first and second segments 30a and 30b within—rather than around—receiver 14 and first tube 18 can allow the receiver and first tube to have relatively large cross-sectional dimensions, while yielding sufficient flexibility at the distal portion of system 10 for the system to navigate through a patient's neurovasculature. For example, outer cross-sectional dimensions 42b and 38b of receiver 14 (when compressed) and first tube 18 can each be greater than or equal to any one of, or between any two of, 0.060″, 0.065″, 0.070″, 0.075″, 0.080″, 0.085″, or 0.090″ (e.g., at least 0.080″), and inner cross-sectional dimensions 42a and 38a of the receiver (when compressed) and first tube can be greater than or equal to any one of, or between any two of, 0.055″, 0.060″, 0.065″, 0.070″, 0.075″, 0.080″, or 0.085″ (e.g., at least 0.075″). Thicker third segment 30c can promote the rigidity of a proximal portion 32a of receiver-supporting element 22, thereby promoting the receiver-supporting element's ability to push receiver 14 and first tube 18 through a patient's vasculature.

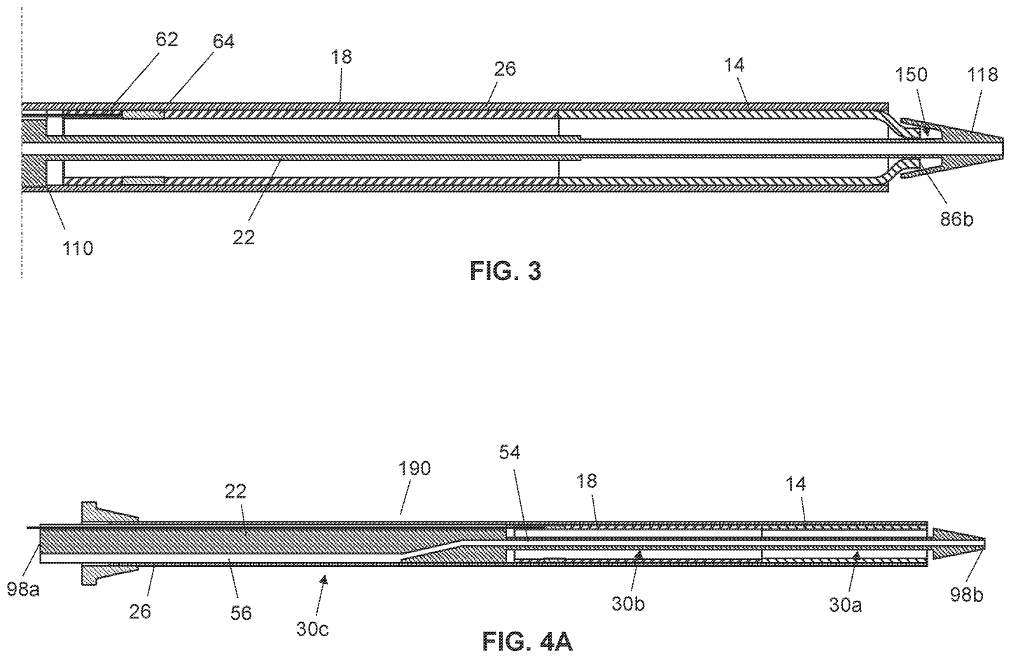

While outer cross-sectional dimensions 46 and 50 of first and second segments 30a and 30b can be the same, as shown in FIG. 3, in some embodiments the second segment's outer cross-sectional dimension can be larger than that of the first segment, such as at least 10%, 15%, 20%, 25%, or 30% larger than the first segment's outer cross-sectional dimension. Additionally, proximal and distal portions 32a and 32b of receiver-supporting element 22 can, but need not, comprise different materials (e.g., different polymers) such that a durometer of the proximal portion is greater than the durometer of the distal portion. Such variations in receiver-supporting element 22's thickness or material composition can each further facilitate the above-described flexibility variations over the length of the receiver-supporting element.

Receiver-supporting element 22 can also include a lumen 54 that extends between its proximal and distal ends 98a and 98b (e.g., through a center of the receiver-supporting element). Lumen 54 can be sized such that a guidewire (e.g., 234, described in further detail below) is receivable therethrough. For example, a cross-sectional dimension 58 (e.g., diameter) of lumen 54 can be greater than or equal to any one of, or between any two of, 0.008″, 0.010″, 0.012″, 0.014″, 0.016″, 0.018″, or 0.020″ (e.g., between 0.010″ and 0.020″). In this manner, system 10 can pass over a guidewire that extends to a thrombus in a patient's neurovasculature, with the guidewire disposed in lumen 54 to provide support therein that can help receiver-supporting element 22 advance receiver 14 and first tube 18 toward the thrombus.

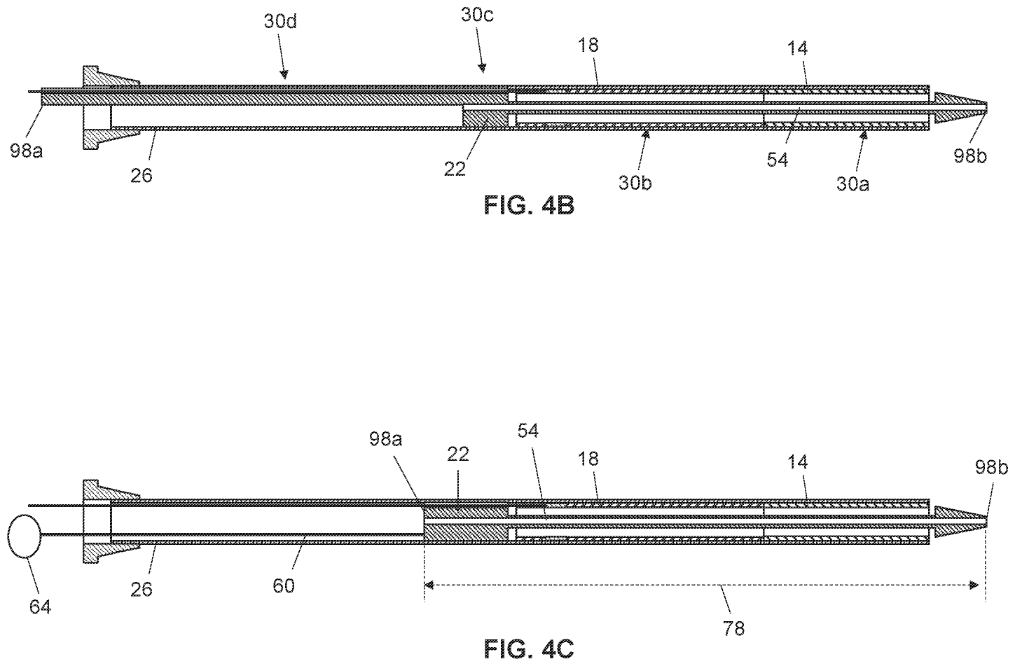

In some embodiments, receiver-supporting element 22 can be configured to permit a rapid-exchange mode of operation in which a shorter guidewire (e.g., a standard guidewire, rather than an exchange-length guidewire) can be used to facilitate single-user operation of system 10. For example, referring to FIGS. 4A and 4B, receiver-supporting element 22′s lumen 54 can extend proximally from distal end 98b through at least first and second segments 30a and 30b, but need not extend to proximal end 98a (e.g., the lumen can terminate in a portion of third segment 30c that is distal of the receiver-supporting element's proximal end). Such a receiver-supporting element 22 can include a pathway by which a guidewire within lumen 54 can exit the receiver-supporting element such that the receiver-supporting element can pass over the guidewire as it is advanced toward a thrombus. To illustrate, as shown in FIG. 4A receiver-supporting element 22 can include an unbounded slit 56 (e.g., in third segment 30c) through which a guidewire extending through its distal end 98b and within lumen 54 can pass to exit receiver-supporting element 22 without passing through its proximal end 98a. Alternatively, as shown in FIG. 4B receiver-supporting element 22 can have a fourth segment 30d (e.g., that is proximal of first, second, and/or third segments 30a-30c) whose outer cross-sectional dimension is smaller than (e.g., less than or equal to 50% of) the maximum outer-cross sectional dimension of the receiver-supporting element 22 (e.g., of the third segment's outer cross-sectional dimension 106) such that a guidewire extending proximally through lumen 54 can exit the lumen without entering the fourth segment.

Referring to FIG. 4C, a rapid-exchange receiver-supporting element 22 can alternatively have a lumen 54 extending between proximal and distal ends 98a and 98b thereof but can be relatively short to permit the use of a shorter guidewire. For example, receiver-supporting element 22′s length 78 can be at least as large as the combined length of first tube 18 and receiver 14 when the receiver is compressed by second tube 26 but less than or equal to any one of, or between any two of, 200%, 190%, 180%, 170%, 160%, 150%, 140%, 130%, or 120% of that combined length. A pusher 60 (e.g., a hypotube or rod) can extend proximally from receiver-supporting element 22′s proximal end 98a to allow the receiver-supporting element to be pushed deeper into a patient's vasculature despite its short length. Pusher 60 can include a handle 64 at a proximal end thereof to facilitate advancement of the pusher and receiver-supporting element. Optionally, a tear line can extend along first, second, and/or third segments 30a, 30b, and 30c through which a guidewire can pass from lumen 54.

Distal end 98b of receiver-supporting element 22 can be defined by an atraumatic distal tip 118 thereof. Tip 118 can, but need not, have a maximum outer cross-sectional dimension (e.g., diameter) 122 that is larger than outer cross-sectional dimensions 46 and 50 of first and second segments 30a and 30b. For example, tip 118′s maximum outer cross-sectional dimension 122 can be greater than or equal to any one of, or between any two of, 110%, 125%, 150%, 175%, 200%, or 300% of each of outer cross-sectional dimensions 46 and 50 of first and second segments 30a and 30b. Tip 118 can thereby provide protection for receiver 14′s distal end 86b as the receiver and first tube 18 are advanced through a patient's vasculature. Tip 118 can also be tapered such that its outer cross-sectional dimension narrows distally along the tip's length, which can facilitate advancement of receiver 14 and first tube 18 and mitigate the risk of damage to a patient's vasculature. As shown in FIG. 3, tip 118 can have a region 150 that can receive distal end 86b of receiver 14, thereby mitigating the resistance caused by the receiver during insertion to facilitate advancement of the receiver and first tube 18. In such embodiments, retraction of receiver-supporting element 22 can cause a portion of receiver 14 that is proximal of its distal end 86b to expand radially outward until the receiver's distal end slips out of region 150, at which point the receiver can be deployed as described in further detail below.

To be able to reach the ICA or MCA (e.g., the M1 segment thereof) from an insertion point at a patient's groin, receiver-supporting element 22 can have a length 78 that is greater than or equal to any one of, or between any two of, 90 cm, 100 cm, 110 cm, 120 cm, 130 cm, 140 cm, or 150 cm (e.g., at least 110 cm). However, as described above in with reference to FIG. 4C, a receiver-supporting element 22 with a pusher 60 extending proximally therefrom can have a shorter length 78, such as a length that is less than or equal to any one of, or between any two of, 60 cm, 55 cm, 50 cm, 45 cm, 40 cm, 35 cm, or 30 cm (e.g., less than or equal to 40 cm). In such embodiments, the combined length of receiver-supporting element 22 and pusher 60 can be greater than or equal to any one of, or between any two of, 90 cm, 100 cm, 110 cm, 120 cm, 130 cm, 140 cm, or 150 cm (e.g., at least 110 cm) to permit ICA or MCA access.

Receiver 14 and first tube 18 can each be shorter than receiver-supporting element 22 such that, when advanced to a thrombus in the ICA or MCA, the receiver and first tube do not extend outside of the patient. Instead, as illustrated in FIGS. 2A and 2B, receiver 14 and first tube 18 can be sized such that the first tube's proximal end 90a is disposable within a distal portion of the larger-cross-sectional-dimension guide catheter 190 (e.g., when the receiver is at a thrombus in the ICA or MCA and the guide catheter's distal end is positioned below the ICA). For example, a length 66 of receiver 14 (e.g., when the receiver is in the expanded state) can be less than or equal to any one of, or between any two of, 20 cm, 18 cm, 16 cm, 14 cm, 12 cm, 10 cm, 8 cm, 6 cm, 4 cm, or 2 cm (e.g., between 2 and 10 cm) and a length 70 of first tube 18 can be less than or equal to any one of, or between any two of, 40 cm, 38 cm, 36 cm, 34 cm, 32 cm, 30 cm, 28 cm, 26 cm, 24 cm, 22 cm, 20 cm, 18 cm, 16 cm, 14 cm, 12 cm, 10 cm, 8 cm, 6 cm, or 4 cm (e.g., between 5 and 30 cm). In this manner, the larger guide catheter 190 can provide a pathway through which a vacuum can be applied for aspiration while receiver 14 and first tube 18 can extend distally from the catheter to reach a thrombus positioned in a portion of the neurovasculature (e.g., in the ICA or MCA) that the catheter, due to its size, may not be able to readily reach.

To facilitate manipulation of receiver 14 and first tube 18 (e.g., to assist the advancement or retraction thereof) while they are disposed within a patient, system 10 can comprise a positioning element 62—such as a push rod or rope (e.g., suture)—connected to and disposed proximal of the first tube. For example, positioning element 62's length 74 can be greater than or equal to any one of, or between any two of, 80 cm, 90 cm, 100 cm, 110 cm, 120 cm, 130 cm, or 140 cm (e.g., at least 90 cm), optionally such that lengths 66, 70, and 74 of receiver 14, first tube 18, and the positioning element are together larger than receiver-supporting element 22's length 78 and/or greater than or equal to any one of, or between any two of, 90 cm, 100 cm, 110 cm, 120 cm, 130 cm, 140 cm, or 150 cm (e.g., at least 110 cm). Positioning element 62′s proximal end 94a can thus be disposed outside of a patient while the receiver and first tube are in the ICA or MCA. Additionally, positioning element 62 can be relatively narrow such that it occupies only a small portion of the lumen of a guide catheter 190 from which receiver 14 and first tube 18 extend such that a thrombus can readily pass through the guide catheter during aspiration. For example, positioning element 62 can have a cross-sectional dimension (e.g., diameter) 66 that is less than or equal to any one of, or between any two of, 0.020″, 0.018″, 0.016″, 0.014″, 0.012″, 0.010″, or 0.008″.

Positioning element 62 can comprise any suitable material to assist pushing and/or pulling of receiver 14 and first tube 18. For example, positioning element 62 can be a rod comprising a metal such as stainless steel, nitinol, and/or the like. Such a metal positioning element 62 can be rigid such that at least some force applied to proximal end 94a thereof will be readily transmitted through the positioning element to first tube 18. In this manner, positioning element 62 can assist receiver-supporting element 22 in advancing receiver 14 and first tube 18 through a patient's vasculature, and can be pulled to withdraw the receiver and first tube after aspiration. Alternatively, positioning element 62 can comprise a non-metal rope, such as a rope comprising a polymer (e.g., aramid). Such a non-metal positioning element 62 can be non-rigid, which can promote system 10′s flexibility; while a non-rigid positioning element may not readily transmit a pushing force to first tube 18 to assist receiver-supporting element 22 during insertion of the system (e.g., because a pushing force may cause deformation of a non-rigid rope), it can be pulled to withdraw receiver 14 and the first tube from the patient's vasculature.

To accommodate positioning element 62, third segment 30c of receiver-supporting element 22 can include a channel 114 (FIG. 1G). Positioning element 62 can extend from first tube 18 and through at least a portion of channel 114 such that the positioning element's proximal end 94a can remain outside of the patient as receiver-supporting element 22 advances receiver 14 and the first tube through the vasculature. Channel 114 can have an unbounded cross-section such that positioning element 62 need not pass through the channel's proximal or distal ends 94a and 94b to enter or exit the channel.

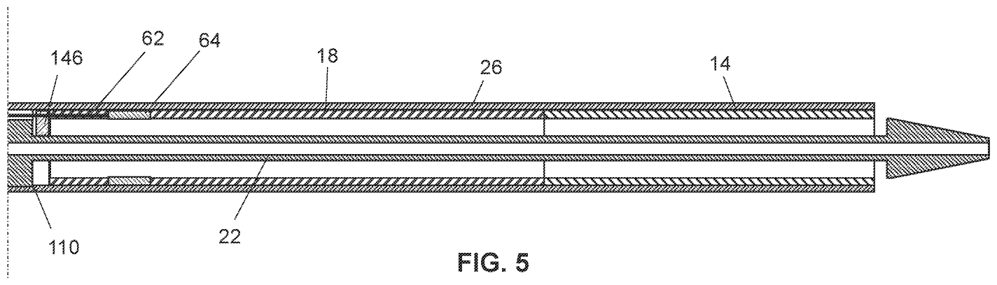

Referring to FIG. 5, in some embodiments system 10 can also comprise a shoulder support 146—such as a piece of material or sheath comprising metal or a polymer—connected to first tube 18's proximal end 90a and, optionally, positioning element 62. Shoulder support 146 can have a thickness (e.g., measured in a radial direction) that is larger than that of first tube 18's wall and can have a durometer higher than that of the first tube. When receiver-supporting element 22 is advanced, its shoulder portion 110 can engage shoulder support 146 such that the force applied on the receiver-supporting element is transmitted to first tube 18 and receiver 14 via the shoulder support. In this manner, shoulder support 146 can mitigate the risk of damage to first tube 18's inner wall when receiver-supporting element 22 advances receiver 14 and the first tube toward the thrombus.

As described above, second tube 26 can contain receiver 14, first tube 18, and receiver-supporting element 22 to facilitate the insertion thereof into a patient's vasculature (e.g., into a guide catheter 190 disposed in the vasculature). To do so, second tube 26's inner cross-sectional dimension 34a (e.g., diameter) can be at least as large as outer cross-sectional dimensions 42b and 106 of first tube 18 and third segment 30c, respectively, such as greater than or equal to any one of, or between any two of, 0.060″, 0.065″, 0.070″, 0.075″, 0.080″, 0.085″, or 0.090″ (e.g., at least 0.080″). At the same time, second tube 26 can be narrow enough to fit within a guide catheter 190 that has sufficient flexibility to facilitate access up to at least the ICA, optionally such that the second tube can access the narrower vessels of a patient's neurovasculature (e.g., in the ICA or MCA). For example, second tube 26's outer cross-sectional dimension 34b can be less than or equal to any one of, or between any two of, 0.095″, 0.090″, 0.085″, 0.080″, 0.075″, 0.070″, or 0.065″ (e.g., less than or equal to 0.085″). In this manner, second tube 26 can fit within a guide catheter 190 having an inner cross-sectional dimension that is less than or equal to any one of, or between any two of, 0.100″, 0.095″, 0.090″, 0.085″, 0.080″, 0.075″, or 0.070″ (e.g., less than or equal to 0.095″) (e.g., the guide catheter can be an 8F catheter having an inner diameter of approximately 0.090″).

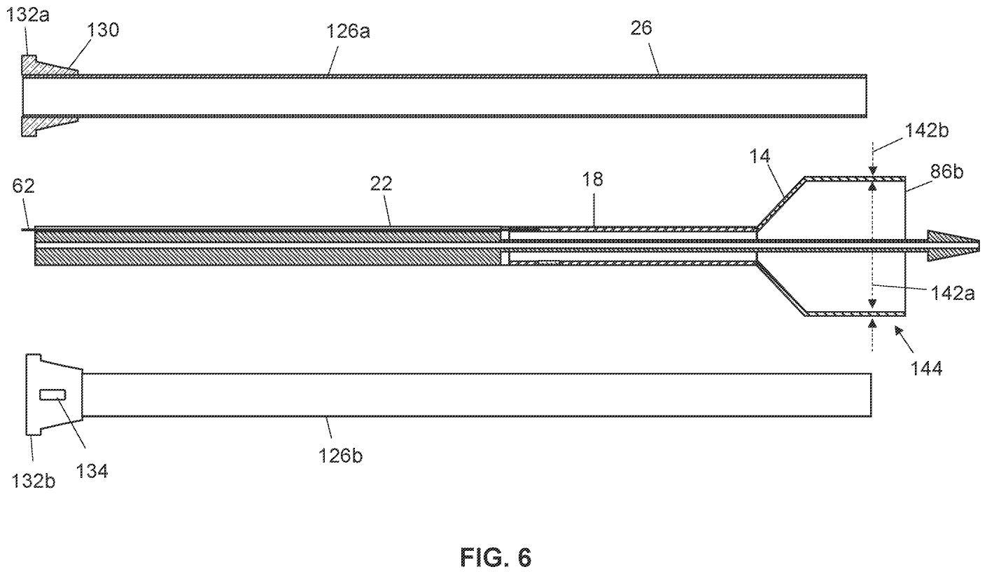

Referring additionally to FIG. 6, to permit deployment of receiver 14 for thrombus ingestion, second tube 26 can be slidable relative to the receiver and first tube 18 and include separable portions (e.g., halves) 126a and 126b, each of which can extend between proximal and distal ends 102a and 102b of the second tube. A force directed distally can be applied to receiver 14 (e.g., by applying a force on receiver-supporting element 22's proximal end 98a and/or on positioning element 62's proximal end 94a) and/or second tube 26 can be retracted proximally (e.g., from its proximal end 102a disposed outside of the patient) such that the receiver's distal end 86b is disposed distally of the second tube's distal end 102b. During and/or after retraction of second tube 26, separable portions 126a and 126b thereof can be separated such that the second tube can be removed from being positioned around other components of system 10. When receiver 14 is deployed, its distal portion 144 can radially expand.

Receiver 14 can be deployed in different manners depending on the proximity between guide catheter 190 and the thrombus. If guide catheter 190 can reach a portion of the neurovasculature in close proximity to the thrombus (e.g., such that a distal end 192b of the guide catheter is within 10 cm, 9 cm, 8 cm, 7 cm, 6 cm, 5 cm, 4 cm, 3 cm, 2 cm, or 1 cm of the thrombus), receiver deployment can occur within a proximal portion the guide catheter such that receiver 14 expands radially to the guide catheter's inner wall. Expanded receiver 14 and first tube 18 can then be advanced through guide catheter 190 at least until the receiver's distal portion 144 is positioned distally of the guide catheter's distal end, allowing the receiver to radially expand further (e.g., to the vessel wall). In such embodiments, length 82 of second tube 26 can be relatively short because unsheathing can occur close to the insertion point; for example, the second tube's length can be less than or equal to any one of, or between any two of, 25 cm, 22 cm, 19 cm, 16 cm, or 13 cm (e.g., less than or equal to 20 cm).

If guide catheter 190 cannot reach a portion of the neurovasculature in close proximity to the thrombus, receiver deployment can occur beyond distal end 192b of the guide catheter. To achieve such deployment, second tube 26 can be advanced toward the thrombus with receiver 14, first tube 18, and receiver-supporting element 22 such that the receiver can remain compressed even when positioned beyond the guide catheter, thereby facilitating delivery thereof. When second tube 26's distal end 102b is close to the thrombus (e.g., within 10 cm, 9 cm, 8 cm, 7 cm, 6 cm, 5 cm, 4 cm, 3 cm, 2 cm, or 1 cm of the thrombus), it can be retracted such that receiver 14 can radially expand (e.g., to the vessel wall). In such embodiments, length 82 of second tube 26 can be relatively long such that it can be in close proximity with the thrombus while its proximal end 102a is disposed outside of a patient; for example, the second tube's length can be greater than or equal to any one of, or between any two of, 90 cm, 100 cm, 110 cm, 120 cm, 130 cm, 140 cm, or 150 cm (e.g., at least 110 cm).

To facilitate separation of second tube 26′s separable portions 126a and 126b, the second tube can include a hub 130 at its proximal end 102a that includes separable hub portions 132a and 132b, each attached to a respective one of the second tube's separable portions. Each of hub portions 132a and 132b can include a wing 134 extending outwardly from hub 130; a distance between the wings' outer ends can be at least 50%, 75%, 100%, 150%, 200%, 300%, or 400% larger than second tube 26's outer cross-sectional dimension 34b. Wings 134 can thus be readily grippable, allowing wings 134 to be pulled apart to cause hub portions 132a and 132b and thus second tube 26's separable portions 126a and 126b to separate. Hub 130 can, but need not, include a seal 138 configured to form a seal around a cylindrical structure passed therethrough (e.g., receiver-supporting element 22), which can mitigate fluid egress when system 10 is advanced to a thrombus.

When in its expanded state, receiver 14's distal portion 144 can have internal and external transverse dimensions (e.g., diameters) 142a and 142b that are larger than those of first tube 18, and the receiver can narrow moving proximally from its distal end (e.g., such that the receiver's internal and external transverse dimensions 42a and 42b at its proximal end 86a are substantially equal to those of the first tube). For example, when fully expanded, receiver 14's internal transverse dimension 142a in its distal portion 144 can be at least 10%, 20%, 30%, 40%, 50%, 60%, 70%, 100%, or 200% larger than each of first tube 18's internal transverse dimension 38a and the receiver's internal transverse dimension 42a at proximal end 86a, such as greater than or equal to any one of, or between any two of, 0.100″, 0.125″, 0.150″, 0.175″, 0.200″, 0.225″, or 0.250″ (e.g., at least 0.125″), and its external transverse dimension 142b can be greater than or equal to any one of, or between any two of, 0.110″, 0.125″, 0.150″, 0.175″, 0.200″, 0.225″, 0.275″, or 0.300″ (e.g., at least 0.175″). As sized, receiver 14's distal portion 144 can radially expand to contact the vessel walls in a patient's neurovasculature, thereby facilitating thrombus ingestion by arresting flow to the thrombus and providing a larger mouth into which the thrombus can be ingested as described in further detail below.

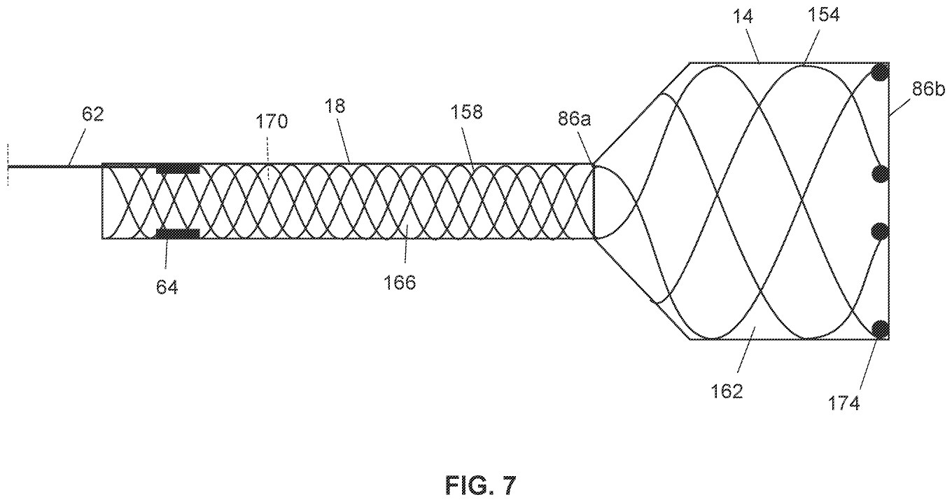

Receiver 14 can have any suitable structure that yields the above-described expandability and permits the receiver to occlude flow between portions of a patient's blood vessel that are proximal and distal of the receiver. Referring to FIG. 7, for example, receiver 14 can comprise a polymer 162 (e.g., a polymeric membrane) that defines an outer surface thereof. Polymer 162 can be liquid-impermeable such that receiver 14, when its distal portion 144 expands and contacts the vessel wall, can impede blood flow to a thrombus while allowing fluid communication between the thrombus, first tube 18, and guide catheter 190 for aspiration. Suitable polymers include polytetrafluoroethylene (PTFE), urethane, silicone, a polyolefin, and/or the like. PTFE, for example, advantageously exhibits low friction with other surfaces and thus facilitates insertion and deployment of receiver 14. To further facilitate delivery of receiver 14, the receiver can include an outer hydrophilic coating to mitigate the resistance between it and other surfaces the receiver makes contact with.

To promote its expandability, receiver 14 can include a frame 154. Frame 154 can be configured to urge radial expansion of receiver 14 when the receiver is radially compressed. As shown, frame 154 comprises a braid; in other embodiments, however, the frame can comprise struts. Suitable materials for frame 154 can include nitinol (i.e., an alloy comprising nickel and titanium), which is superelastic such that the frame can regain its original shape when a mechanical load exerted thereon is released, and/or stainless steel. Bonding between frame 154 and polymer 162 can be achieved in a variety of ways such that at least a portion of the frame is surrounded by at least a portion of the polymer. As one example, frame 154 can be embedded in polymer 162. Alternatively, receiver 14 can comprise a second polymeric membrane (e.g., comprising the same material of polymer 162) that defines the receiver's inner wall and is adhered to polymer 162 such that frame 154 is disposed between the two membranes.

Receiver 14 can also comprise one or more radiopaque markers 174, which can be embedded in polymer 162. Radiopaque marker(s) 174 can inhibit the passage of X-rays therethrough and thus can be viewed via fluoroscopy when receiver 14 is disposed in a patient. For example, each radiopaque marker 174 can comprise tantalum or platinum. Radiopaque marker(s) 174 can thereby aid a physician in determining the position of receiver 14 in a patient's vasculature during insertion and deployment thereof. At least one radiopaque marker 174 can be disposed closer to receiver 14′s distal end 86b than to its proximal end 86a, such as within 1 cm, 0.9 cm, 0.8 cm, 0.7 cm, 0.6 cm, 0.5 cm, 0.4 cm, 0.3 cm, 0.2 cm, or 0.1 cm of the distal end. Such distally-positioned radiopaque marker(s) 174 can assist a physician in determining receiver 14's position relative to a thrombus such that the receiver can be positioned adjacent thereto to achieve adequate engagement for aspiration.

First tube 18 can be structured such that it can engage with receiver-supporting element 22 and transmit the force to receiver 14 when the receiver-supporting element advances the receiver and first tube through a patient's vasculature. For example, as shown, first tube 18 can also comprise a polymer 166 (e.g., defining the outer surface thereof), such as nylon, polyether block amide, polyurethane, and/or the like. First tube 18's polymer 166 can be reinforced with a frame 158, which can comprise a braid, a coil, struts, and/or the like that comprises metal. Frame 158 of first tube 18 can be in direct contact with receiver 14's frame 154. To illustrate, first tube 18's frame 158 can be integral with receiver 14's frame 154, which can promote the strength of the connection between the receiver and first tube. Alternatively, a portion of receiver 14's frame 154 can be embedded in first tube 18's polymer 166, which similarly can facilitate the connection between the receiver and the first tube.

As shown, positioning element 62 can be connected to first tube 18 via a ring 64, which can comprise, for example, a metal such as stainless steel. At least a portion of ring 64 can be surrounded by at least a portion of first tube 18′s polymer 166, with the ring positioned closer to the first tube's proximal end 90a than to its distal end 90b. Ring 64 can thus provide a strong connection between first tube 18 and positioning element 62.

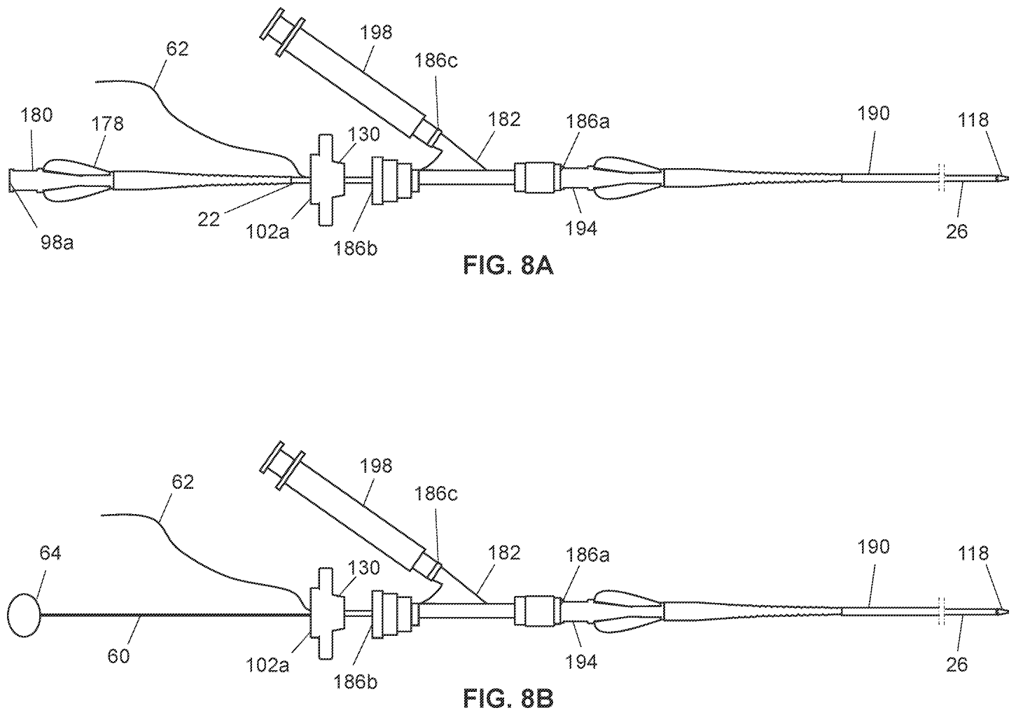

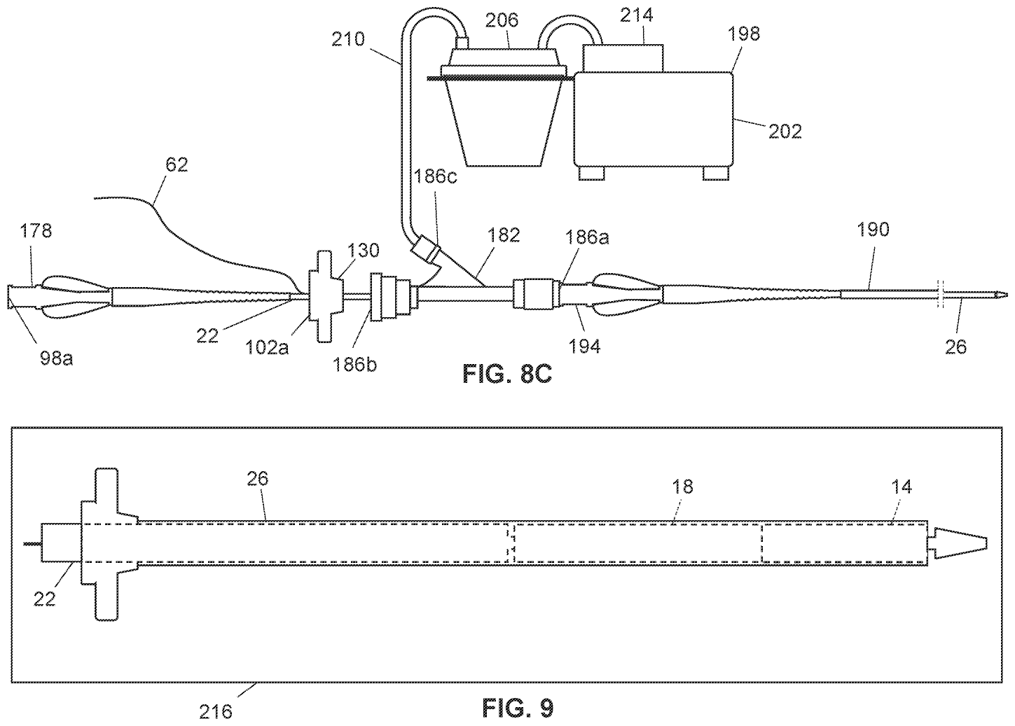

Referring to FIGS. 8A-8C, a multi-port adapter 182 can allow system 10 to interface with guide catheter 190 and a vacuum source 198 (e.g., a syringe (FIGS. 8A-8B) or a vacuum pump (FIG. 8C)) for aspiration. Multi-port adapter 182 can comprise at least three ports 186a-186c to do so. First port 186a can be configured to be coupled to a proximal connector 194 of guide catheter 190 such that a lumen of multi-port adapter 182 is in fluid communication with the guide catheter's lumen. Second port 186b can be configured to permit second tube 26—with receiver 14, first tube 18, and receiver-supporting element 22 disposed therein—to pass therethrough into the lumen of multi-port adapter 182 and through the lumen of guide catheter 190 when the guide catheter's proximal fitting 194 is coupled to first port 186a. As shown in FIG. 8A, receiver-supporting element 22 can include a hub 178 and a proximal connector 180 that are proximate of second tube 26's hub 130 and second port 186b, which can be connected to syringe to allow flushing prior to use. Alternatively, as shown in FIG. 8B, in embodiments in which receiver-supporting element 22 is relatively short and has a pusher 60 extending proximally therefrom, the pusher can extend out of second port 186b and second tube 26's hub 130; in such embodiments, flushing can be achieved through the receiver-supporting element's distal end 98b. With system 10 positioned through second port 186b, receiver 14 and first tube 18 can be advanced to a thrombus in a patient's neurovasculature as described above. Additionally, second tube 26 can be withdrawn through second port 186b during deployment of receiver 14, as can receiver-supporting element 22 prior to aspiration.

For aspiration, multi-port adapter 108 can comprise a third port 186c that can be coupleable to a vacuum source 198. Third port 186c can have a luer lock for achieving such a vacuum source connection. When vacuum source 198 is coupled to third port 186c, it can be in fluid communication with the lumen of multi-port adapter 182 and thus with the lumen of guide catheter 190. Vacuum source 198 can thus apply a vacuum to guide catheter 190 by lowering the pressure at third port 186c, thereby drawing the thrombus into receiver 14 and through first tube 18 and the guide catheter. To promote efficient application of the vacuum and mitigate blood leakage out of multi-port adapter 182, second port 186b can be closed during aspiration such that fluid cannot flow therethrough. For example, second port 186b can be configured to seal around a cylindrical structure positioned therethrough (e.g., can comprise a Tuohy-Borst adapter) such that the second port can form a seal around positioning element 62 after second tube 26 and receiver-supporting element 22 are withdrawn.

Vacuum source 198 can comprise any suitable device by which a vacuum can be applied to guide catheter 190 to draw a thrombus into the deployed receiver 14 and through first tube 18 and the guide catheter for removal. For example, as shown in FIGS. 8A and 8B vacuum source 198 can comprise a syringe that, optionally, has a barrel configured to hold greater than or equal to any one of, or between any two of, 40 mL, 50 mL, 60 mL, 70 mL, or 80 mL of fluid. The relatively small negative pressure differential that a syringe can yield between guide catheter 190's proximal end and receiver 14's distal end 86b during aspiration can be sufficient for thrombus ingestion and removal due at least in part to the relatively large cross-sectional areas of receiver 14's mouth and throat. For example, the magnitude of the pressure differential vacuum source 198 can apply between receiver 14's mouth and a proximal end of guide catheter 190 can be less than or equal to any one of, or between any two of, 180, 160, 140, 120, or 100 mm Hg.

Alternatively, and as shown in FIG. 8C, vacuum source 198 can comprise a vacuum pump, which can include a pumping unit 202 (e.g., having a motor) and a container 206 in fluid communication with the pumping unit such that the pumping unit can draw a vacuum on the container. Container 206 can in turn be coupled to multi-port adapter 182's third port 186c via a tube 210 such that pumping unit 202 is in fluid communication with and thus can apply a vacuum at guide catheter 190's proximal end via the container, which can receive fluids drawn from a patient's vasculature during aspiration. Pumping unit 202 can be configured to control the pressure at guide catheter 190's proximal end (e.g., with a regulator 214) to yield a sufficient pressure differential for thrombus removal.

Referring to FIG. 9, any of the present systems can be included in a kit. In such a kit, self-expanding receiver 14, first tube 18, and receiver-supporting element 22 can already be positioned at least partially within second tube 26 such that the receiver and first tube are ready for insertion into a patient, thereby allowing prompt treatment. As shown, self-expanding receiver 14, first tube 18, receiver-supporting element 22, and second tube 26 can be positioned in a sealed container 216 such that they remain sterile.

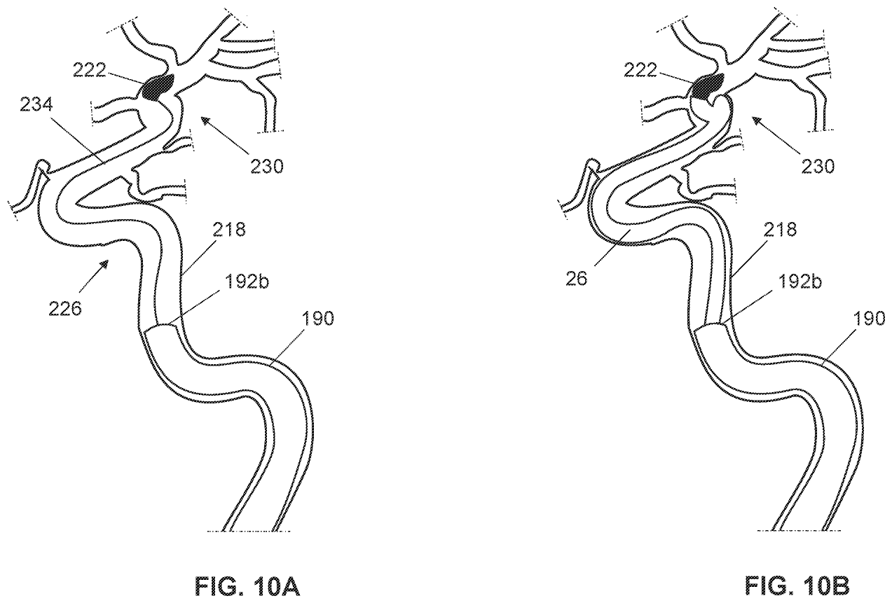

Turning to FIGS. 10A and 10B, some of the present methods of removing a thrombus (e.g., 222) (e.g., a red thrombus or a white thrombus) comprise advancing a guidewire (e.g., 234) through vasculature (e.g., 218) of a patient and advancing a guide catheter (e.g., 190) over the guidewire (FIG. 10A). As described above, the guidewire and catheter can be inserted into the patient's vasculature at the groin. The guidewire can extend to the thrombus to facilitate advancement of the catheter through the patient's vasculature.

With the guide catheter disposed in the patient's vasculature, some methods comprise advancing a system (e.g., 10) (e.g., any of those described above) over the guidewire and into the catheter (FIG. 10B). As described above, the system can include a self-expanding receiver (e.g., 14), a first tube (e.g., 18) connected to the receiver, a positioning element (e.g., 62) connected to the first tube, a receiver-supporting element (e.g., 22), and a second tube (e.g., 26). The receiver and first tube can be positioned around first and second segments (e.g., 30a and 30b), respectively, of the receiver-supporting element and the second tube can positioned around at least a portion of the receiver. Additionally, as described above, the positioning element can be disposed in at least a portion of a channel (e.g., 114) in a third segment (e.g., 30c) of the receiver-supporting element.

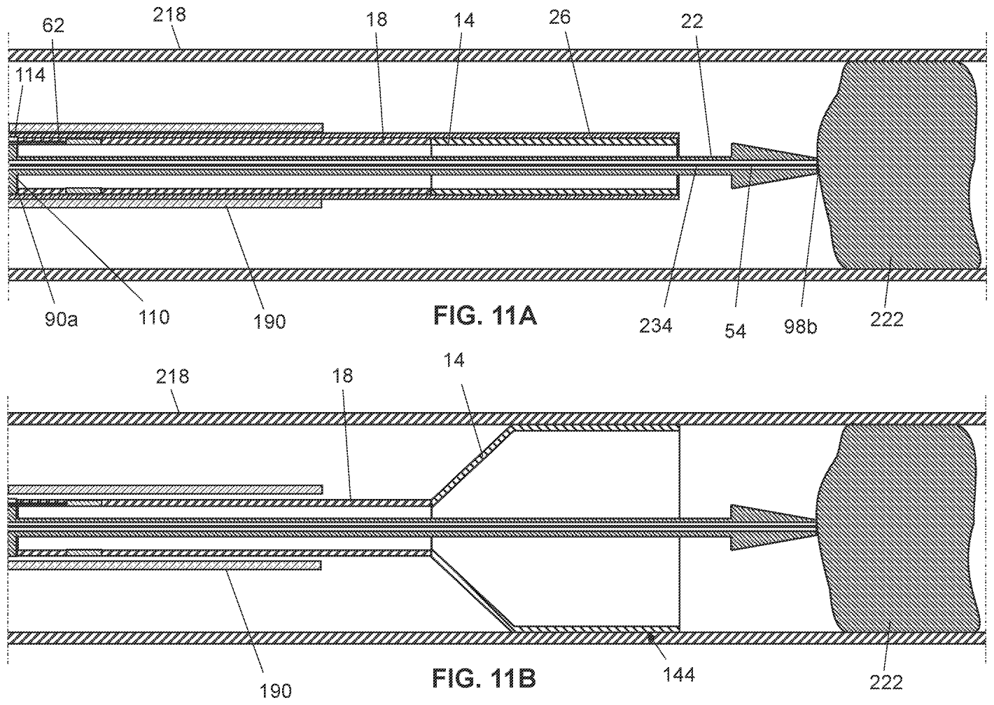

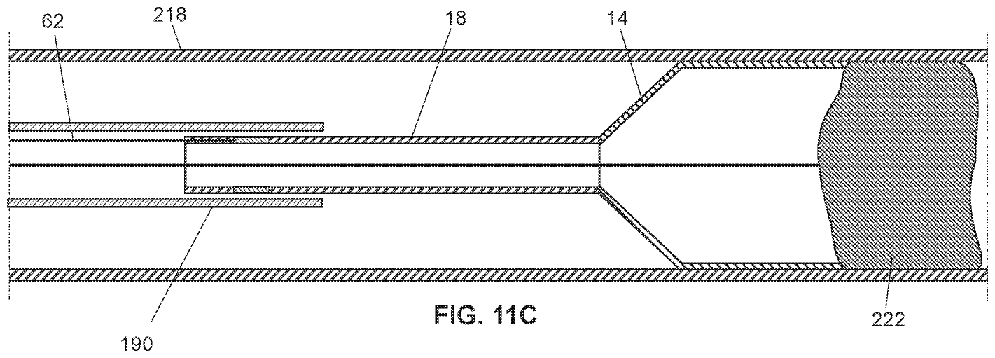

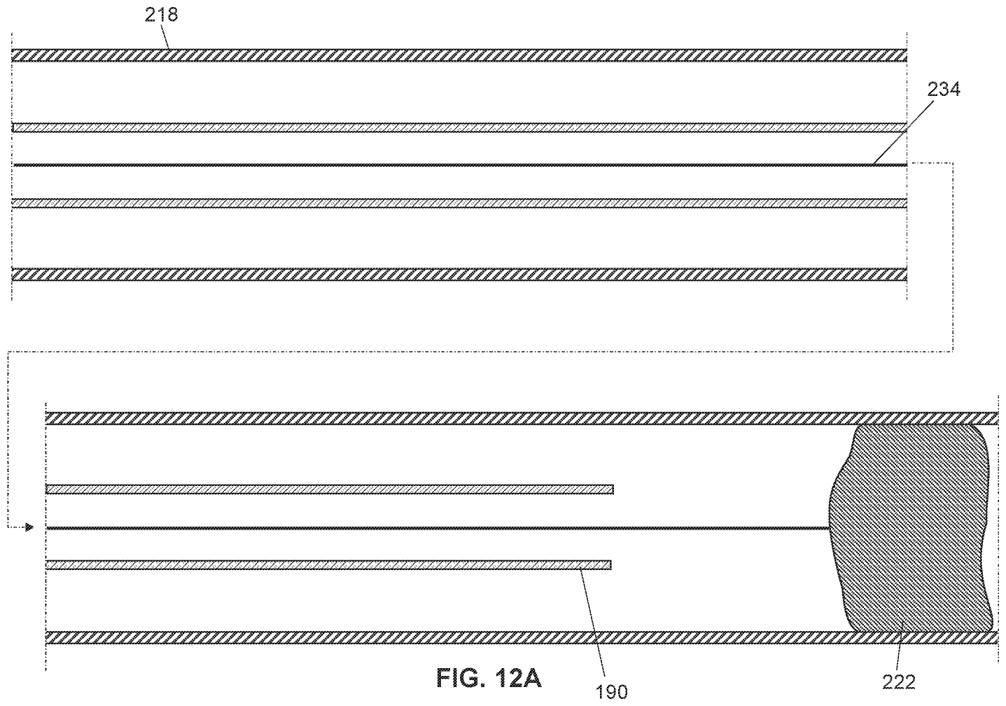

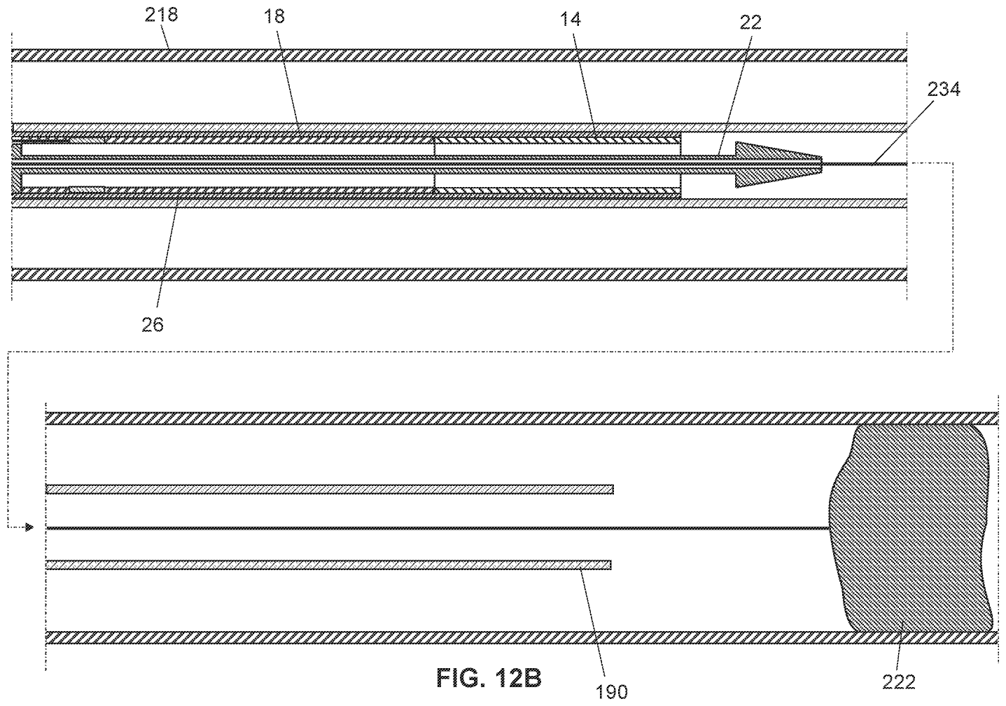

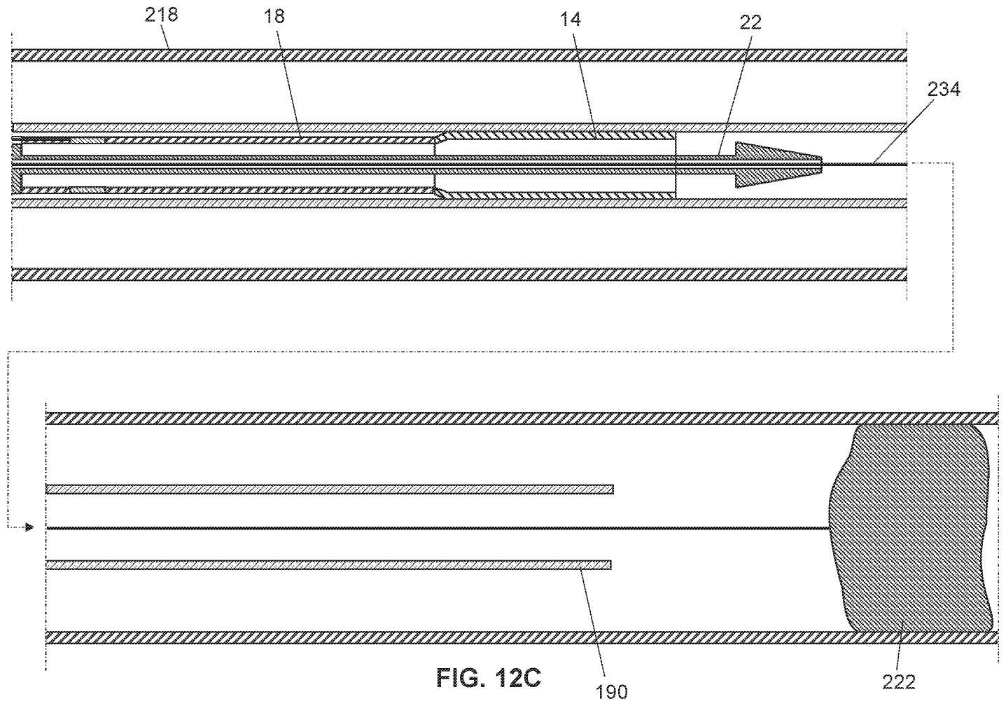

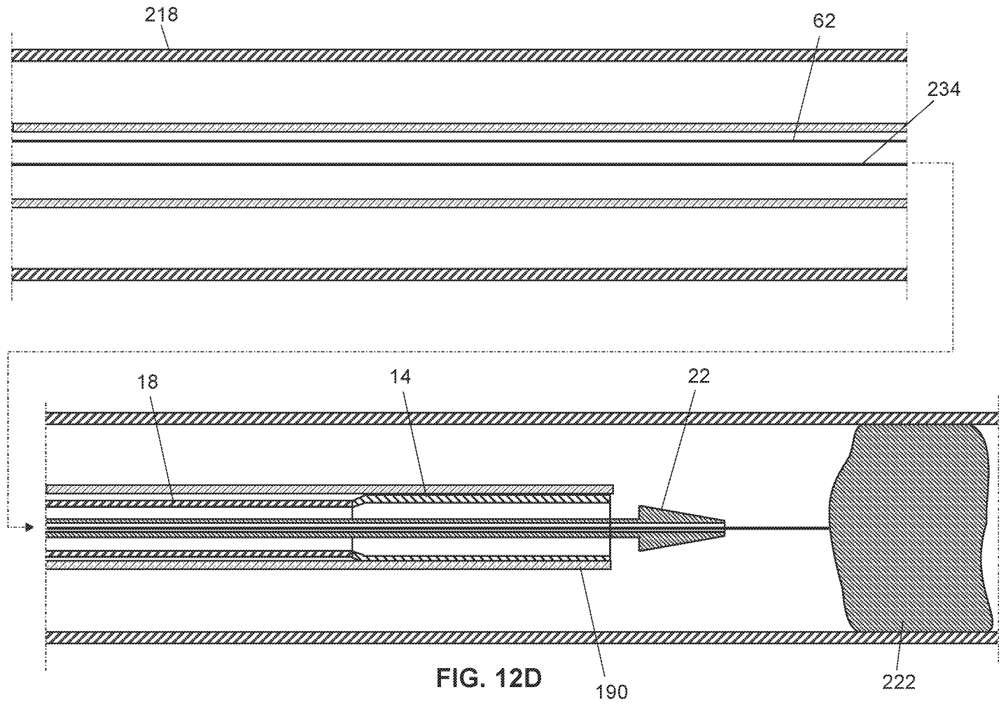

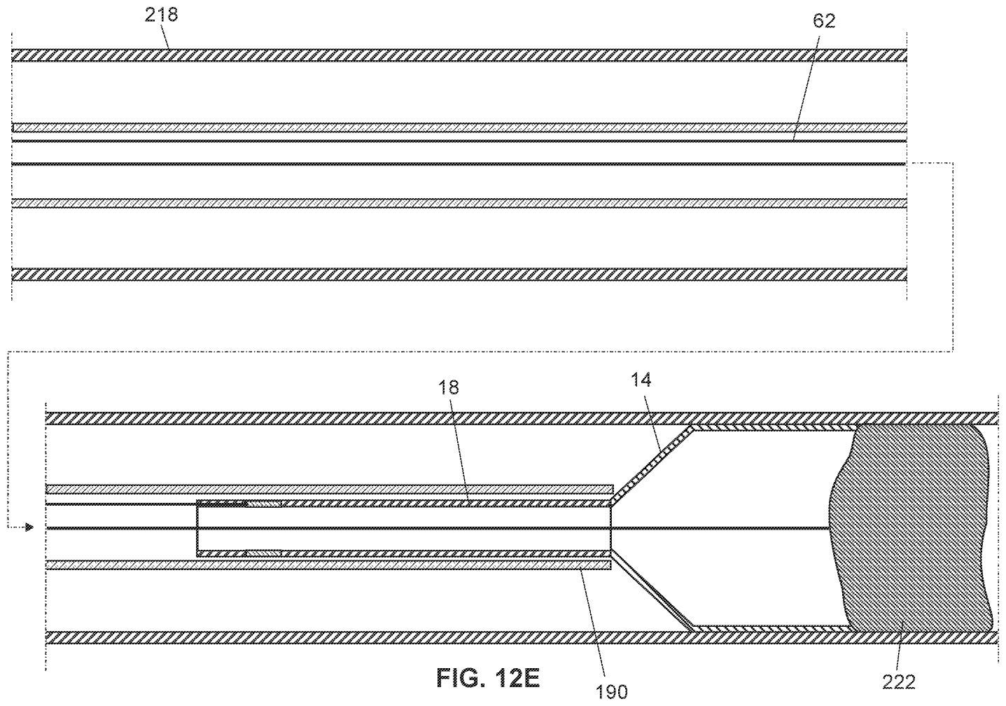

Referring additionally to FIGS. 11A-11C and 12A-12E, some methods comprise advancing the receiver-supporting element, the self-expanding receiver, the first tube, and the positioning element within the catheter and toward the thrombus, either with (FIGS. 11A-11C) or without (FIGS. 12A-12E) the second tube. To do so, a distally-urging force can be applied to the receiver-supporting element (e.g., at its proximal end (e.g., 98a)) such that it engages the first tube (e.g., with the shoulder portion (e.g., 110) contacting the first tube's proximal end (e.g., 90a)) to cause these components to move distally. If the positioning element is rigid (e.g., a pusher rod), a force can also be applied thereto (e.g., at its proximal end (e.g., 94a)) to assist advancement of the system. As shown, the guidewire can be positioned within a lumen (e.g., 54) of the receiver-supporting element such that the receiver-supporting element, first tube, and receiver are positioned around the guidewire, which can facilitate advancement thereof to the thrombus. These components can be advanced until at least a portion of the first tube is positioned in a distal portion of the guide catheter and at least a distal portion (e.g., 144) (up to and including all) of the receiver is distal of the guide catheter's distal end (e.g., 192b) such that the receiver can be deployed for thrombus aspiration. When deployed, the receiver's distal portion can expand to contact the patient's vessel (FIGS. 11B and 12E).

As explained above, and referring particularly to FIGS. 11A-11C, the second tube can be advanced with the receiver-supporting element, the self-expanding receiver, the first tube, and the positioning element to facilitate access to a thrombus positioned in a portion of the vasculature that the guide catheter may not be able to readily reach. For example, as shown in FIG. 10B, the guide catheter can be advanced up to the ICA (e.g., 226), such as within 5 cm, 4 cm, 3 cm, 2 cm, or 1 cm of the ICA, and the second tube can be advanced until its distal end (e.g., 102b) is distal of the guide catheter's distal end, such as in the ICA or MCA (e.g., 230) (e.g., the M1 segment thereof) where the thrombus is located. The receiver can be positioned in a constrained orientation in the second tube (e.g., in a distal portion thereof) such that they are advanced to near the thrombus together, e.g., such that the distal ends of the second tube and receiver are each positioned within 5 cm, 4 cm, 3 cm, 2 cm, or 1 cm of the thrombus (FIG. 11A). In this manner, the receiver can remain compressed when advanced to the thrombus such that it can be readily delivered thereto for deployment.

To deploy the receiver when the second tube's distal end is advanced beyond the guide catheter, the second tube can be retracted (e.g., by pulling a proximal portion of the second tube) while a force is applied to the positioning element (e.g., when the positioning element is rigid, such as when it is a pusher rod) and/or to the receiver-supporting element (e.g., whether or not the positioning element is rigid) (FIG. 11B). The second tube can thus move proximally relative to the receiver such that the receiver is unsheathed. The receiver can be positioned such that the distal end (e.g., 86b) thereof is near or in contact with the thrombus (e.g., by adjusting its position by applying a force on the positioning element and/or the receiver-supporting element), which can facilitate aspiration. The second tube can be moved proximally until it is withdrawn from the guide catheter, and the separable portions (e.g., 126a and 126b) thereof can also be separated as described above such that the second tube is removed from being around the guidewire and the receiver-supporting element.

With the receiver deployed, the receiver-supporting element can be withdrawn such that its distal end (e.g., 98b) is proximal of the first tube's proximal end (FIG. 11C). The receiver-supporting element can continue to be retracted and removed from the catheter such that the pathway defined by the receiver, first tube, and catheter is available for aspiration.

Referring particularly to FIGS. 12A-12E, if the receiver-supporting element, receiver, first tube, and positioning element are to be advanced within the catheter without the second tube, once the system is advanced over the guidewire and into the catheter (FIGS. 12A and 12B) (e.g., when positioned in a proximal portion of the guide catheter), the second tube can be retracted out of the catheter (FIG. 12C) and the separable portions thereof can be separated. Retraction of the second tube can occur in the manner described above (e.g., by pulling the second tube while applying a force to the receiver-supporting element and/or to the positioning element). As shown, because the receiver remains within the catheter when the second tube is retracted, the receiver can expand radially to contact the catheter's inner wall. The guide catheter can provide adequate compression of the receiver for advancement thereof, and without the extra rigidity imposed by the second tube, advancing the receiver-supporting element, receiver, first tube, and positioning element within the guide catheter can be easier.

With the second tube retracted out of the catheter and separated, the receiver-supporting element, receiver, first tube, and positioning element can be advanced within the catheter in the manner described above (e.g., by applying a distally-urging force to the positioning element (if rigid) and/or to the receiver-supporting element) (FIG. 12D). The receiver can be deployed by advancing the receiver at least partially beyond the guide catheter's distal end such that the distal portion of the receiver expands further to contact the patient's vessel. Because this expansion can occur as the receiver exits the guide catheter, deployment in this manner may be suitable when the guide catheter's distal end can be positioned in close proximity to the thrombus as explained above. As in the delivery-in-the-second-tube technique, the receiver-supporting element can be withdrawn such that the receiver-supporting element's distal end is proximal of the first tube's proximal end, and can be removed from the catheter to render the pathway defined by the receiver, first tube, and guide catheter available for aspiration.

Regardless of the manner of deployment, the receiver's maximum uncompressed external transverse dimension can be larger than the vessel's internal transverse dimension (e.g., diameter) such that the expanded distal portion can exert sufficient pressure on the blood vessel to occlude blood flow therein. For example, the receiver's distal portion can exert a pressure that is greater than or equal to any one of, or between any two of, 40 kPa, 50 kPa, 60 kPa, 70 kPa, 80 kPa, 90 kPa, 100 kPa, or 110 kPa (e.g., between 50 and 100 kPa) on the vessel wall.

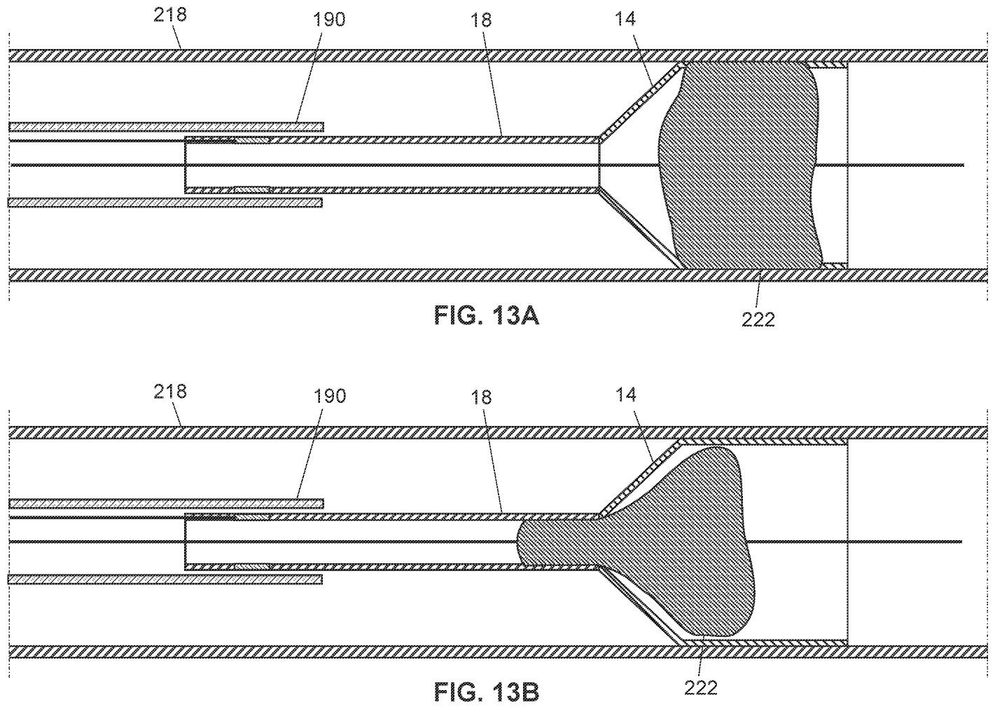

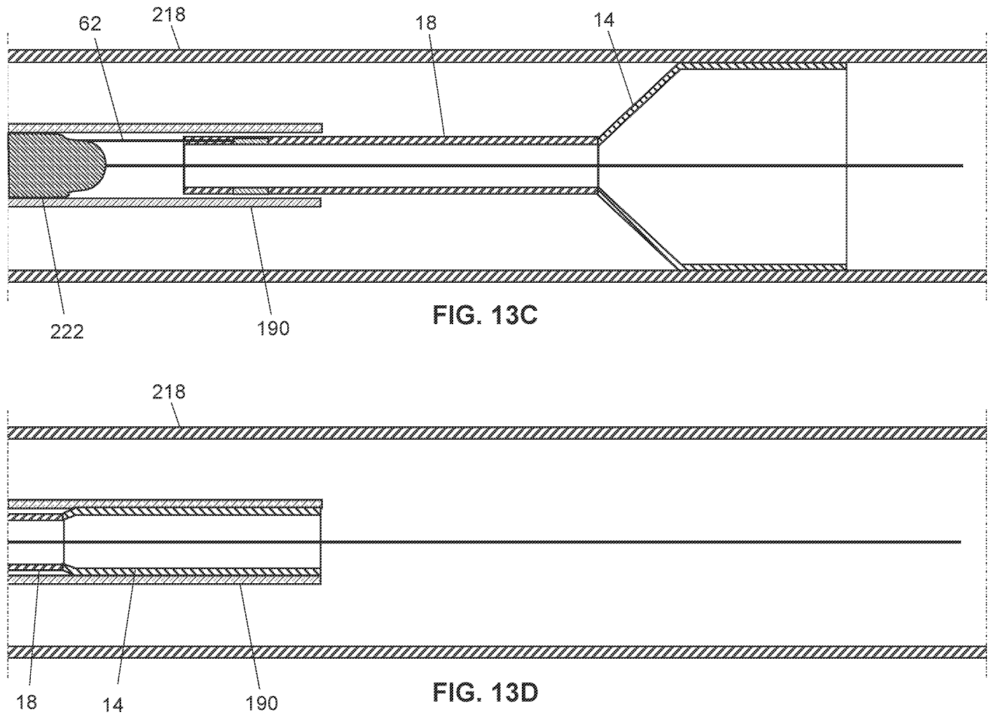

Referring to FIGS. 13A-13D, some methods comprise applying a vacuum to the catheter (e.g., in any of the manners described above, such as with a syringe or vacuum pump). As a result, pressure at the catheter's proximal end (e.g., 192a) can be reduced, yielding a negative pressure differential between the receiver's distal and the catheter's proximal end that can help cause the thrombus to aspirate into the receiver (FIG. 13A). Because the receiver's distal portion is expanded to the vessel wall, such ingestion can readily occur. Additionally, and referring to FIGS. 13B and 13C, application of the vacuum can cause the thrombus to aspirate through the receiver and into the first tube and catheter. As shown, the receiver's narrowing transition section and the first tube's large inner cross-sectional dimension (e.g., which is permitted by the receiver-supporting element as described above) facilitate deformation and compression of the thrombus such that the thrombus can enter the first tube. This ingestion can occur even if the thrombus is a white thrombus that is more resistant to compression than a red thrombus. The vacuum can continue to draw the ingested thrombus through the first tube, into the guide catheter, and out of the guide catheter's proximal end. If a vacuum pump is used for aspiration, recanalization can be confirmed when a change in pump pressure occurs. In some methods, the receiver and the first tube can be moved proximally relative to the guide catheter by pulling on the positioning element (FIG. 13D) such that they can be removed.

In some procedures, the thrombus may not aspirate into the first tube when the vacuum is applied, even with the relatively large receiver throat. When this occurs, to remove the thrombus the receiver, first tube, and catheter can be withdrawn from the patient with the thrombus disposed in the receiver. Alternatively, the receiver and first tube can be withdrawn into the guide catheter (e.g., with the positioning element) while the vacuum is applied to the guide catheter, which may allow thrombus ingestion into the first tube and/or the guide catheter for removal.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A system for use in blood clot removal, the system comprising:
a receiver-supporting element having a distal end, a first segment that includes a first segment cross-sectional outer dimension, a second segment that includes a second segment cross-sectional outer dimension, a proximal end, and a lumen extending proximally from the distal end and through at least the first and second segments of the receiver-supporting element;
a self-expanding receiver including a frame and a polymer, at least a portion of the receiver positioned around at least a portion of the first segment;
a first tube connected to the receiver, at least a portion of the first tube positioned around at least a portion of the second segment; and
a second tube positioned around at least a portion of the receiver, the second tube having separable portions.

2. The system of claim 1, wherein:
the receiver-supporting element also has a channel; and
the system further comprises a positioning element connected to the first tube, a portion of the positioning element positioned in at least a portion of the channel.

3. The system of claim 2, wherein the first segment cross-sectional outer dimension and the second segment cross-sectional outer dimension are the same.

4. The system of claim 2, wherein the second segment cross-sectional outer dimension is greater than the first segment cross-sectional outer dimension.

5. The system of claim 2, wherein the receiver-supporting element also has an atraumatic distal tip having a cross-sectional outer dimension larger than the first segment cross-sectional outer dimension.

6. The system of claim 5, wherein the atraumatic distal tip has a region in which a distal end of the receiver is positioned.

7. The system of claim 6, wherein the receiver-supporting element also has a third segment positioned proximal of the first and second segments, the third segment including the channel and having a cross-sectional outer dimension larger than the second segment cross-sectional outer dimension.

8. (canceled)

9. The system of any of claim 2, further comprising a shoulder support connected to both the first tube and the positioning element.

10. The system of any of claim 7, wherein the receiver-supporting element also has a hub, and a proximal end of the second tube is distal of the proximal end of the receiver-supporting element.

11-23. (canceled)

24. The system of claim 10, wherein the second tube also has a valve seal comprising separable valve seal portions.

25-27. (canceled)

28. The system of claim 24, wherein the receiver-supporting element, the self-expanding receiver, the first tube, the positioning element, and the second tube are positioned in a sealed container.

29. A method for use in blood clot removal, the method comprising:
advancing a guidewire through vasculature of a patient;
advancing a catheter over the guidewire, and
advancing a system over the guidewire and into the catheter, the system comprising:
a receiver-supporting element;
a self-expanding receiver positioned around a first portion of the receiver-supporting element;
a first tube connected to the receiver and positioned around a second portion of the receiver-supporting element;
a positioning element connected to the first tube; and
a second tube positioned around at least a portion of the receiver, the second tube having separable portions.

30. The method of claim 29, wherein at least a portion of the positioning element is positioned in at least a portion of a channel positioned in a third portion of the receiver- supporting element.

31. The method of claim 30, further comprising advancing the receiver-supporting element, the self-expanding receiver, the first tube, and the positioning element within the catheter while the second tube is not advanced over the guidewire.

32. The method of claim 31, further comprising:
retracting the second tube out of the catheter; and
separating the separable portions of the second tube and removing the second tube from being positioned around the guidewire.

33. The method of claim 32, wherein the positioning element is rigid and further comprising after the separating, applying force to the receiver-supporting element and the positioning element so as to advance the receiver-supporting element, the self-expanding receiver, the first tube, and the positioning element within the catheter.

34. The method of claim 32, further comprising after the separating, applying force to the receiver-supporting element so as to advance the receiver-supporting element, the self-expanding receiver, the first tube, and the positioning element within the catheter.

35. (canceled)

36. The method of claim 33, further comprising deploying the self-expanding receiver such that a distal portion of the self-expanding receiver expands and contacts a vessel of the patient.

37. The method of claim 36, further comprising withdrawing the receiver-supporting element such that a distal end thereof is proximal of a proximal end of the first tube.

38. (canceled)

39. The method of claim 37, further comprising pulling on the positioning element so that the first tube and the self-expanding receiver move proximally relative to the catheter.

40-48. (canceled)
